(19) United States
(12) Patent Application Publication
(10) Pub. No.: US 2012/0263669 A1
(43) Pub. Date: Oct. 18, 2012

(54) PRODUCTS FOR KERATIN FIBERS, CONTAINING AT LEAST ONE SPECIAL CATIONIC POLYMER A HAVING VINYLIMIDAZOLE STRUCTURAL UNITS AND AT LEAST ONE SPECIAL ALKOXYLATED CATIONIC SURFACTANT

(76) Inventors: Burkhard Mueller, Hamburg (DE); Matthias Schweinsberg, Hamburg (DE)

(21) Appl. No.: 13/537,122

(22) Filed: Jun. 29, 2012

Related U.S. Application Data
(63) Continuation of application No. PCT/EP2010/069143, filed on Dec. 8, 2010.

(30) Foreign Application Priority Data
Dec. 29, 2009 (DE) 10 2009 055 354.1

(51) Int. Cl. A61K 8/81; A61Q 5/06

(52) U.S. Cl. 424/70.15

(57) ABSTRACT

Agents for treating keratin-containing fibers, particularly human hair, containing in a cosmetically acceptable carrier (a) at least one cationic polymer having at least one structural unit of formula (I), at least one structural unit of formula (II), and at least one structural unit of formula (III),

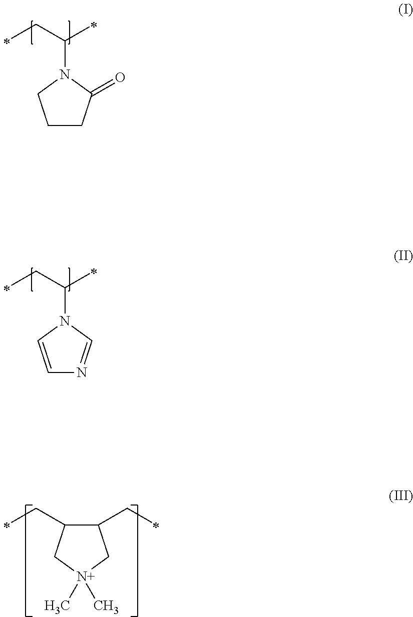

and (b) at least one cationic surfactant of formula (IV),

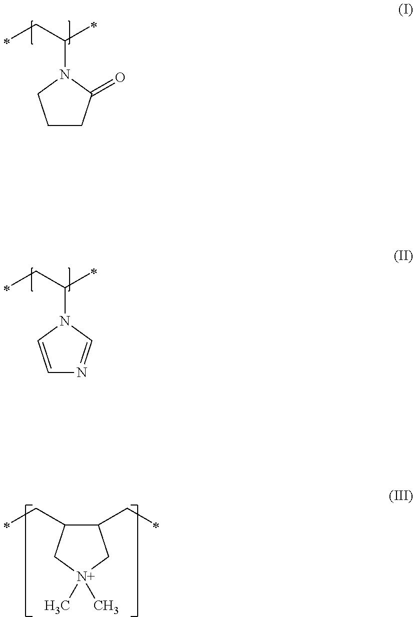

wherein x and y are independently an integer number greater than 0, R is a (C₈ to C₂₀) alkyl group or a (C₈ to C₂₀) alkenyl group, R′ is a *—(CH₂CH₂O)_{z}H group wherein z is an integer number greater than 0, a (C₈ to C₂₀) alkyl group or a (C₈ to C₂₀) alkenyl group, and X⁻ is a physiologically acceptable anion. The agents are gentle and can be used as hair care agents having outstanding styling properties, particularly in the form of an aerosol hair spray or aerosol hair foam.

### CROSS REFERENCE TO RELATED APPLICATIONS

The present application is a continuation of International Application No. PCT/EP2010/069143 filed 8 Dec. 2010, which claims priority to German Patent Application No. 10 2009 055 354.1, filed 29 Dec. 2009, both of which are incorporated herein by reference.

### BACKGROUND OF THE INVENTION

The present invention relates to agents for hair treatment containing a combination of at least one special cationic polymer having vinylimidazole structural units and at least one special alkoxylated cationic surfactant, use of those agents for the temporary shaping and/or care of keratin-containing fibers, and aerosol hair sprays/foams based on those agents.

Keratin-containing fibers are understood in principle to be all animal hair, for example, wool, horsehair, angora hair, fur, feathers and products or textiles manufactured therefrom. Keratinic fibers are, however, preferably human hair.

An attractive-looking hairstyle is today regarded as an essential element of a well-groomed exterior. The latest fashion trends mean that for many hair types, hairstyles that are regarded as fashionable can only be constructed or maintained for a lengthy period of up to several days using active fixing agents. Therefore, hair treatment agents that provide a permanent or temporary shaping of the hair have an important role to play. Temporary shaping effects which offer good hold without adversely affecting the healthy appearance of the hair, such as its shine, can be achieved, for example, using hair sprays, hair waxes, hair gels, hair foams, blow-drying, etc.

Corresponding agents for temporary shaping conventionally contain synthetic polymers as the shaping component. Preparations containing a dissolved or dispersed polymer can be applied to the hair using propellants or a pump mechanism. Hair gels and hair waxes in particular, however, are generally not applied directly to the hair, but are distributed in the hair using a comb or the hands.

An important property of an agent for temporarily shaping keratinic fibers, also referred to herein as a styling agent, consists in giving the treated fibers in the created shape the strongest possible hold. If the keratinic fibers are human hair, this property is also described as a strong styling hold or a high degree of hold of the styling agent. Styling hold is substantially determined by the nature and amount of synthetic polymer that is used, although additional ingredients of the styling agent can also have an influence.

In addition to having a high degree of hold, styling agents also have to satisfy a series of further requirements. These include properties of the hair, properties of the individual formulation (e.g., properties of the foam, gel or sprayed aerosol), and properties relating to the handling of the styling agent, with particular importance being attached to the properties of the hair. Mention can be made in particular of moisture resistance, low tackiness and a balanced conditioning effect. As far as possible, a styling agent should also be universally suitable for all hair types and be gentle on the hair and skin.

In order to meet these diverse requirements, a large number of synthetic polymers for use in styling agents have been developed. The polymers can be divided into cationic, anionic, non-ionic and amphoteric fixing polymers. The polymers ideally form a polymer film when applied to the hair, giving the hairstyle a strong hold while remaining sufficiently flexible not to break under stress. If the polymer film is too brittle, film flakes or residues are formed which detach when the hair is moved and give the impression that the user of the corresponding styling agent has dandruff.

Developing styling agents that offer a combination of all desired properties continues to present difficulties. This is particularly true for the combination of strong hold and an easy, uniform application onto the keratin-containing fibers.

The present invention therefore provides an agent for the temporary shaping and/or care of keratinic fibers that is distinguished by a high degree of hold or a good caring effect, is gentle on the skin, and in particular is extremely easy to handle during application on the keratin-containing fibers.

### SUMMARY OF THE INVENTION

Surprisingly it has now been found that this can be achieved through a combination of a special cationic polymer and a special alkoxylated cationic surfactant. If formulated as hair foam, the compositions achieved with this combination have particularly good foam parameters.

The present invention thus firstly provides an agent for treating keratin-containing fibers, particularly human hair, containing in a cosmetically acceptable carrier—
(a) at least one cationic polymer comprising at least one structural unit of formula (I), at least one structural unit of formula (II) and at least one structural unit of formula (III),

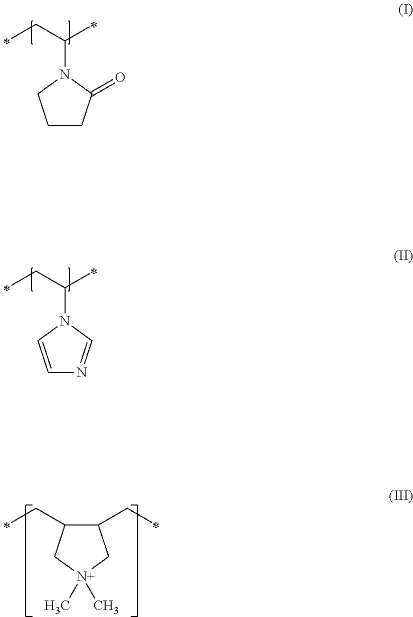

and
(b) at least one cationic surfactant of formula (IV),

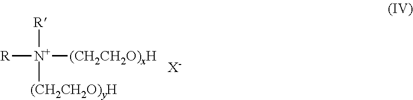

wherein
x and y are, independently of one another, an integer number greater than 0,
R is a (C₈ to C₂₀) alkyl group or a (C₈ to C₂₀) alkenyl group,
R′ is a *—(CH₂CH₂O)_{z}H group wherein z is an integer number greater than 0, a (C₈ to C₂₀) alkyl group or a (C₈ to C₂₀) alkenyl group, and
X′ is a physiologically acceptable anion.

In the above formulae and all subsequent formulae, a chemical bond marked with the symbol * represents a free valence of the corresponding structural fragment.

### DETAILED DESCRIPTION OF THE INVENTION

At least one physiologically acceptable anion, such as chloride, bromide, hydrogen sulfate, methyl sulfate, ethyl sulfate, tetrafluoroborate, phosphate, hydrogen phosphate, dihydrogen phosphate or p-toluenesulfonate, triflate, serves to offset the positive polymer charge in the agent according to the invention.

Examples of (C₈ to C₃₀) alkyl groups according to the invention are octyl (capryl), decyl (caprinyl), dodecyl (lauryl), tetradecyl (myristyl), hexadecyl (cetyl), octadecyl (stearyl), eicosyl (arachyl), docosyl (behenyl). Examples of (C₈ to C₂₀) alkenyl groups according to the invention are oleyl, linolyl, linolenyl.

In the cationic polymer (a), the structural units of formulae (I), (II) and (III) can be randomly distributed or can be present in the form of polymeric or oligomeric blocks, such as blocks of poly[diallyldimethylammonium] units (particularly poly[diallyldimethylammonium chloride] units) and blocks of poly[N-vinylpyrrolidone/N-vinylimidazole] units. Blocks of poly[N-vinylpyrrolidone/N-vinylimidazole] units consist of structural units of the above formulae (I) and (II), which are randomly distributed within the block.

Preferably, cationic polymer (a) has an average molecular weight M_{w} (weight average) from 50,000 to 250,000 g/mol, particularly from 75,000 to 200,000 g/mol.

Such a cationic polymer (a) thus includes at least one structural unit of formula (Block-1) and at least one structural unit of formula (Block-2)—

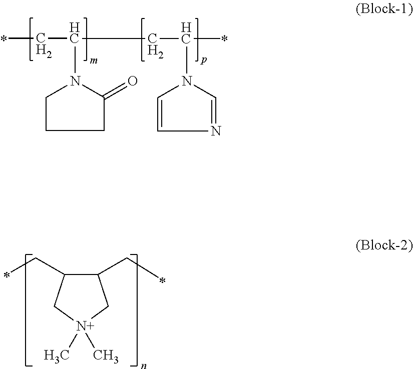

wherein
m and p are an integer greater than 0, the building blocks within the structural unit of formula (Block-1) are randomly distributed, and
n is an integer greater than 1, particularly greater than 10.

Cationic polymer (a) is preferably present in agents according to the invention in an amount from 0.01 wt. % to 20 wt. %, more preferably 0.01 wt. % to 10.0 wt. %, even more preferably from 0.1 wt. % to 3 wt. %, based on total weight of the agent.

A preferred cationic polymer (a) is a copolymer of N-vinylpyrrolidone, N-vinylimidazole and a diallydimethylammonium compound (particularly diallyldimethylammonium chloride). Such copolymers are known under the INCI name Polyquaternium-87 and are sold, for example, by BASF SE under the trade name Luviquat Sensation in the form of an aqueous solution containing 27.5 wt. % of active substance. Preferably, the agents contain at least one polymer with the INCI name Polyquaternium-87.

In addition to cationic polymer (a), agents according to the invention contain at least one cationic surfactant (b) according to formula (IV) above.

The cationic surfactant of formula (IV) is preferably present in agents according to the invention in an amount from 0.01 wt. % to 20 wt. %, more preferably 0.01 wt. % to 10.0 wt. %, even more preferably from 0.2 wt. % to 2 wt. %, based on total weight of the agent.

Preferably, the cationic surfactant of formula (IV) has a molecular weight from 550 to 2500 g/mol, more preferably from 600 to 1000 g/mol.

R according to formula (IV) is preferably dodecyl, tetradecyl, hexadecyl or octadecyl.

Preferably, R′ according to formula (IV) is a *—(CH₂CH₂O)_{z}H group, wherein z is an integer greater than 0.

X⁻ according to formula (IV) is preferably chloride, phosphate or dihydrogen phosphate.

It is also preferable if, according to formula (IV), x and y (and also z if R′ is the *—(CH₂CH₂O)_{z}H group) are independently an integer number chosen from 1, 2, 3, 4, 5, 6, 7, 8, 9, and 10.

It is further preferable according to the invention for the sum of x and y according to formula (IV) (or, if R′ is a *—(CH₂CH₂O)_{z}H group, the sum of x and y and z) to be a number from 3 to 20, particularly a number from 5 to 15.

A particularly preferred cationic surfactant has the formula (IVa)—

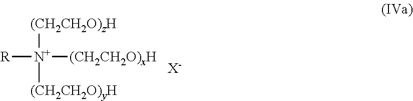

wherein
x, y and z are independently an integer number greater than 0, and the sum of (x+y+z) is a number from 3 to 20, particularly from 5 to 15,
R is a (C₈ to C₂₀) alkyl group or a (C₈ to C₂₀) alkenyl group, and
X⁻ is a physiologically acceptable anion.

A most particularly preferred cationic surfactant is the tris(oligooxyethyl)alkylammonium dihydrogen phosphate salt having a molecular weight of 780 g/mol and the INCI name Quaternium-52, which is sold, for example, by Cognis under the trade name Dehyquart® SP as a 50 wt. % aqueous solution.

A most particularly preferred agent according to the invention for treating keratin-containing fibers, particularly human hair, contains in a cosmetically acceptable carrier—

### (a) Polyquaternium-87 and

### (b) Quaternium-52.

The aforementioned preferred embodiments, particularly the stated amounts, are also preferred for this embodiment with necessary alterations.

Agents according to the invention can optionally additionally contain at least one fixing polymer. These additional polymers are different from cationic polymer (a). The optionally added fixing polymers are preferably cationic and/or non-ionic.

Preferred agents for treating keratin-containing fibers, particularly human hair, contain in a cosmetically acceptable carrier—
(a) at least one cationic polymer having at least one structural unit of formula (I), at least one structural unit of formula (II), and at least one structural unit of formula (III),

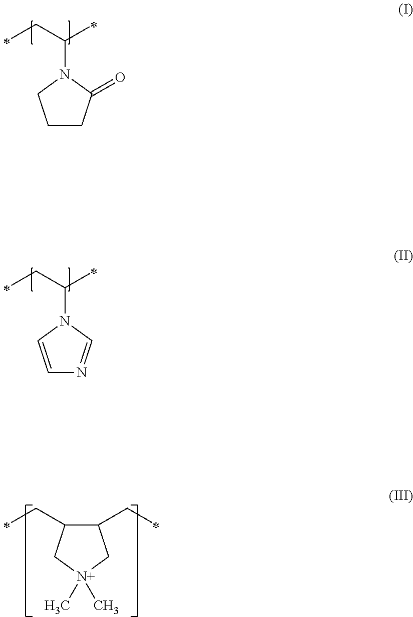

(b) at least one cationic surfactant of formula (IVa)

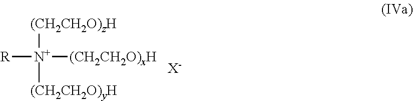

wherein
x, y and z are independently an integer number greater than 0, and the sum of (x+y+z) is a number from 3 to 20, particularly from 5 to 15,
R is a (C₈ to C₂₀) alkyl group or a (C₈ to C₂₀) alkenyl group, and
X⁻ is a physiologically acceptable anion,
and
(c) at least one (particularly cationic and/or non-ionic) fixing polymer.

Fixing polymers aid in maintaining and/or establishing volume and fullness in the hairstyle as a whole. These polymers are simultaneously also film-forming polymers and are therefore generally typical substances for hair treatment agents used for shaping, such as hair fixing agents, hair foams, hair waxes, and hair sprays. Film formation may be entirely localized here and may bond only a few fibers together.

The curl retention test is frequently used as a test method for the fixing effect of a polymer.

Film-forming polymers refer to polymers which leave behind a continuous film on the skin, hair or nails when they dry. Such film formers can be used in a wide range of cosmetic products such as face masks, make-up, hair fixing agents, hair sprays, hair gels, hair waxes, hair tonics, shampoos or nail varnishes. Such polymers are particularly preferred which have an adequate solubility in water or water/alcohol mixtures in order for them to be present in the agent in completely dissolved form. The film-forming polymers can be of synthetic or natural origin.

Film-forming polymers also include polymers which, when used in a 0.01 to 20 wt. % aqueous, alcoholic or aqueous-alcoholic solution, are capable of depositing a transparent polymer film on the hair.

Agents according to the invention can additionally contain at least one cationic fixing polymer.

Cationic fixing polymers have at least one structural unit containing at least one permanently cationized nitrogen atom. Permanently cationized nitrogen atoms refer to nitrogen atoms bearing a positive charge and thus forming a quaternary ammonium compound. Quaternary ammonium compounds are mostly produced by reacting tertiary amines with alkylating agents, such as methyl chloride, benzyl chloride, dimethyl sulfate, dodecyl bromide, and ethylene oxide. Depending on the tertiary amine used, the following groups are known in particular: alkyl ammonium compounds, alkenyl ammonium compounds, imidazolinium compounds and pyridinium compounds.

Preferred agents according to this embodiment contain cationic film-forming and/or cationic fixing polymers in an amount from 0.1 wt. % to 20.0 wt. % preferably 0.2 wt. % to 10.0 wt. %, more preferably 0.5 wt. % to 5.0 wt. %, based on total weight of the agent.

Cationic fixing polymers can be chosen from cationic, quaternized cellulose derivatives.

As a general rule, cationic, quaternized celluloses bearing more than one permanent cationic charge in a side chain are advantageous within the meaning of the embodiment.

Of these, cationic cellulose derivatives which are produced by reacting hydroxyethyl cellulose with a dimethyldiallylammonium reactant (particularly dimethyldiallylammonium chloride), optionally in the presence of further reactants, should be mentioned. Of these cationic celluloses, cationic celluloses with the INCI name Polyquaternium-4 are particularly suitable, and are sold, for example, by National Starch under the names Celquat® H 100, Celquat® L 200.

Further suitable cationic fixing polymers comprise at least one structural unit of formula (I), at least one structural unit of formula (VI), and optionally at least one structural unit of formula (V)—

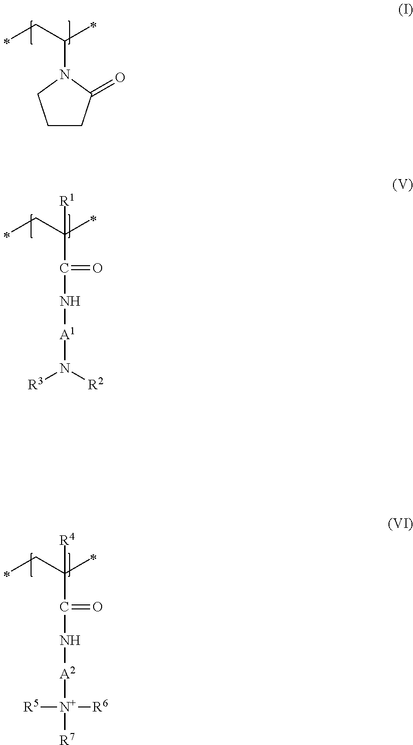

wherein
R¹ and R⁴ are independently a hydrogen atom or a methyl group,
A¹ and A² are independently an ethane-1,2-diyl, propane-1,3-diyl or butane-1,4-diyl group,
R², R³, R⁵ and R⁶ are independently a (C₁ to C₄) alkyl group, and
R⁷ is a (C₈ to C₃₀) alkyl group.

All possible physiologically acceptable anions, such as chloride, bromide, hydrogen sulfate, methyl sulfate, ethyl sulfate, tetrafluoroborate, phosphate, hydrogen phosphate, dihydrogen phosphate or p-toluene sulfonate, triflate, serve to offset the positive charge of monomer (VI).

Suitable commercially obtainable compounds include—
copolymers of diethyl sulfate-quaternized dimethylaminoethyl methacrylate with N-vinylpyrrolidone with the INCI name Polyquatemium-11, under the names Gafquat® 440, Gafquat® 734, Gafquat® 755 (all ISP) and Luviquat PQ 11 PN (BASF SE);
copolymers of methacryloylaminopropyl lauryl dimethylammonium chloride with N-vinylpyrrolidone and dimethylaminopropyl methacrylamide having the INCI name Polyquaternium-55, under the trade names Styleze® W-10, Styleze® W-20 (ISP); and
copolymers of methacryloylaminopropyl lauryl dimethylammonium chloride with N-vinylpyrrolidone, N-vinylcaprolactam and dimethylaminopropyl methacrylamide having the INCI name Polyquaternium-69, under the trade name Aquastyle® 300 (ISP).

Preferred agents for treating keratin-containing fibers, particularly human hair, contain in a cosmetically acceptable carrier—
(a) at least one cationic polymer having at least one structural unit of formula (I), at least one structural unit of formula (II), and at least one structural unit of formula (III),

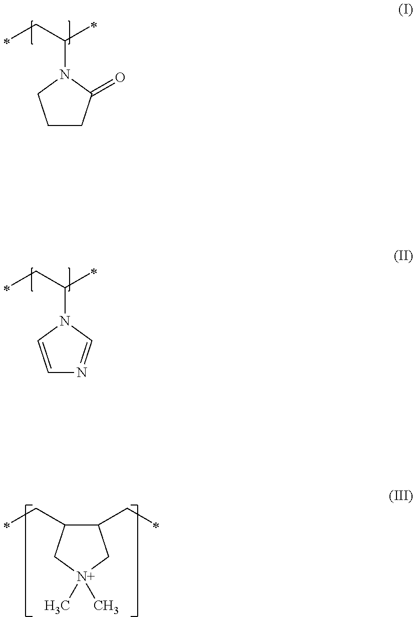

(b) at least one cationic surfactant of formula (IVa),

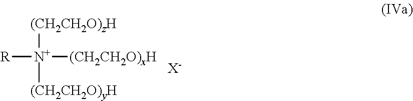

wherein
x, y and z are independently an integer number greater than 0, and the sum of (x+y+z) is a number from 3 to 20, particularly from 5 to 15,
R is a (C₈ to C₂₀) alkyl group or a (C₈ to C₂₀) alkenyl group, and
X⁻ is a physiologically acceptable anion;
and
(c) at least one cationic fixing polymer comprising at least one structural unit of formula (I), at least one structural unit of formula (VI), and optionally at least one structural unit of formula (V),

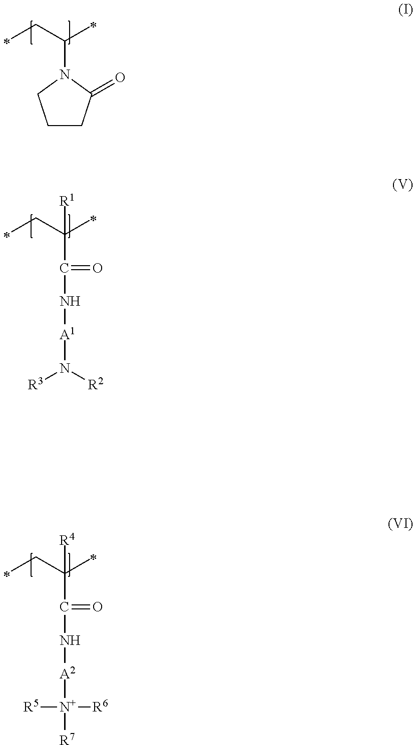

wherein
R¹ and R⁴ are independently a hydrogen atom or a methyl group,
A¹ and A² are independently an ethane-1,2-diyl, propane-1,3-diyl or butane-1,4-diyl group,
R², R³, R⁵ and R⁶ are independently a (C₁ to C₄) alkyl group, and
R⁷ is a (C₈ to C₃₀) alkyl group.

Furthermore, cationic film-forming and/or cationic fixing copolymers serving as film-forming and/or fixing polymers chosen from cationic polymers containing at least one structural unit having a permanently cationized nitrogen atom which can preferably be used according to the embodiment are those comprising at least one structural element of formula (M1)—

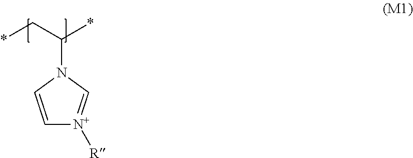

wherein R″ is a (C₁ to C₄) alkyl group, particularly a methyl group, and that additionally have at least one further cationic and/or non-ionic structural element.

Regarding the offsetting of the positive polymer charge, the details given above apply.

In the context of this embodiment, the agent preferably contains as cationic film-forming and/or cationic fixing polymers at least one copolymer (c1) additionally comprising, in addition to at least one structural element of formula (Ml), a structural element of formula (I)—

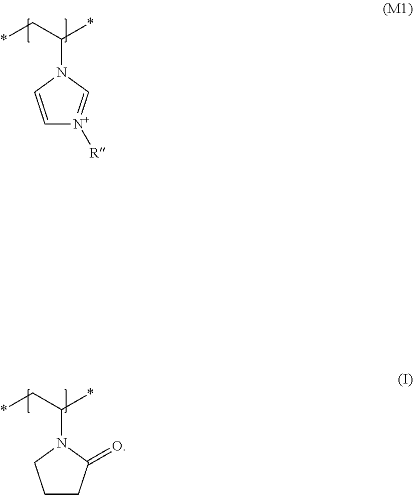

wherein R″ is a (C₁ to C₄) alkyl group, particularly a methyl group.

All possible physiologically acceptable anions, such as chloride, bromide, hydrogen sulfate, methyl sulfate, ethyl sulfate, tetrafluoroborate, phosphate, hydrogen phosphate, dihydrogen phosphate or p-toluenesulfonate, triflate, serve to offset the positive polymer charge of copolymer (c1).

Most particularly preferred cationic film-forming and/or cationic fixing polymers as copolymers (c1) contain 10 to 30 mol %, preferably 15 to 25 mol %, and particularly 20 mol % of structural units according to formula (M1), and 70 to 90 mol %, preferably 75 to 85 mol % and particularly 80 mol % of structural units according to formula (I).

It is particularly preferable for copolymers (c1) to contain in the copolymer, in addition to polymer units resulting from the incorporation of the cited structural units according to formula (M1) and (I), a maximum of 5 wt. %, preferably a maximum of 1 wt. %, of polymer units resulting from the incorporation of other monomers. Copolymers (c1) are preferably exclusively made up of structural units of formula (M1) with R″=methyl and (I) and can be described by general formula (Poly1)—

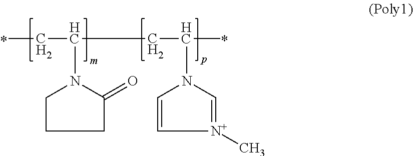

wherein m and p vary based on the molar mass of the polymer and are not intended to suggest that these are block copolymers. Structural units of formulae (M1) and (I) can instead be randomly distributed in the molecule.

If a chloride ion is used to offset the positive charge of the polymer of formula (Poly1), these N-methyl vinylimidazole/vinylpyrrolidone copolymers are referred to under INCI nomenclature as Polyquaternium-16 and are available, for example, from BASF under the trade names Luviquat® Style, Luviquat® FC 370, Luviquat® FC 550, Luviquat® FC 905 and Luviquat® HM 552.

If a methosulfate is used to offset the positive charge of the polymer of formula (Poly1), these N-methyl vinylimidazole/vinylpyrrolidone copolymers are referred to under INCI nomenclature as Polyquaternium-44 and are available, for example, from BASF under the trade name Luviquat® UltraCare.

Particularly preferred agents according to the invention of this embodiment contain a copolymer (c1), particularly of the formula (Poly1), having molar masses within a defined range. Preferred agents here are those wherein copolymer (c1) has a molar mass of 50 to 400 kDa, preferably 100 to 300 kDa, more preferably 150 to 250 kDa and particularly 190 to 210 kDa.

Agents according to the invention can also contain, in addition to copolymer(s) (c1) or in its or their place, copolymers (c2) which, as additional structural units starting from copolymer (c1), have structural units of formula (VII)—

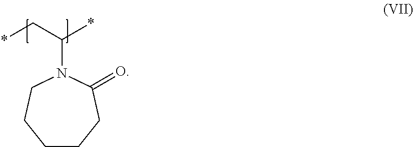

Further particularly preferred agents according to the invention of this embodiment thus contain as cationic film-forming and/or cationic fixing polymer at least one copolymer (c2) containing at least one structural unit according to formula (M1-a), at least one structural unit according to formula (I), and at least one structural unit according to formula (VII)—

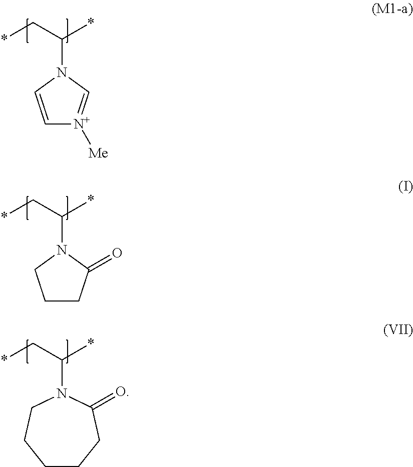

Here, too, it is particularly preferable in the context of this embodiment for copolymers (c2) to contain, in addition to polymer units resulting from the incorporation of the cited structural units according to formula (M1-a), (I) and (VII) in the copolymer, a maximum of 5 wt. %, preferably a maximum of 1 wt. %, of polymer units resulting from the incorporation of other monomers. Copolymers (c2) are preferably exclusively made up of structural units of formulae (M1-a), (I) and (VII) and can be described by the general formula (Poly2)—

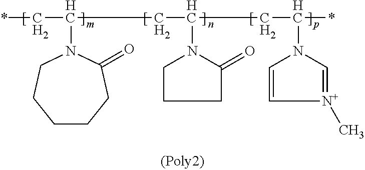

wherein m, n and p vary according to the molar mass of the polymer and are not intended to suggest that these are block copolymers. Structural units of this formula can instead be randomly distributed in the molecule.

All possible physiologically acceptable anions, such as chloride, bromide, hydrogen sulfate, methyl sulfate, ethyl sulfate, tetrafluoroborate, phosphate, hydrogen phosphate, dihydrogen phosphate or p-toluenesulfonate, triflate, serve to offset the positive polymer charge of component (c2).

If a methosulfate is used to offset the positive charge of the polymer of formula (Poly2), such N-methyl vinylimidazole/vinylpyrrolidone/vinylcaprolactam copolymers are referred to under INCI nomenclature as Polyquaternium-46 and are available, for example, from BASF under the trade name Luviquat® Hold.

Most particularly preferred copolymers (c2) contain 1 to 20 mol. %, preferably 5 to 15 mol. % and particularly 10 mol % of structural units according to formula (M1-a), 30 to 50 mol %, preferably 35 to 45 mol % and particularly 40 mol % of structural units according to formula (I), and 40 to 60 mol %, preferably 45 to 55 mol % and particularly 60 mol % of structural units according to formula (VII).

Particularly preferred agents according to the invention of this embodiment contain a copolymer (c2) having molar masses within a defined range. Preferred agents are those wherein copolymer (c2) has a molar mass of 100 to 1000 kDa, preferably 250 to 900 kDa, more preferably 500 to 850 kDa and particularly 650 to 710 kDa.

As film-forming cationic and/or fixing cationic polymer, agents according to the invention can also contain, in addition to copolymer(s) (c1) and/or (c2) or in its or their place, copolymers (c3) which have structural units according to formulae (M1-a) and (I) as well as further structural units from the group of vinylimidazole units and from the group of acrylamide and/or methacrylamide units.

Further particularly preferred agents of this embodiment contain as additional cationic film-forming and/or cationic fixing polymer at least one copolymer (c3) containing at least one structural unit according to formula (M1-a), at least one further structural unit according to formula (I), at least one further structural unit according to formula (VIII), and at least one further structural unit according to formula (IX)—

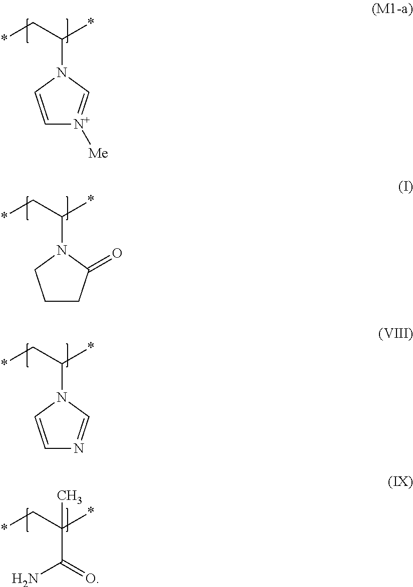

Here, too, it is particularly preferable for copolymers (c3) to contain in the copolymer, in addition to polymer units resulting from the incorporation of the cited structural units according to formula (M1-a), (I), (VIII) and (IX), a maximum of 5 wt. %, preferably a maximum of 1 wt. % of polymer units resulting from the incorporation of other monomers. Copolymers (c3) preferably exclusively comprise structural units of formula (M1-a), (I), (VIII) and (IX) and can be described by the general formula (Poly3)—

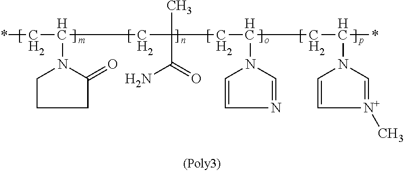

wherein m, n, o and p vary according to the molar mass of the polymer and are not intended to suggest that these are block copolymers. Structural units of formulae (M1-a), (I), (VIII) and (IX) can instead be randomly distributed in the molecule.

All possible physiologically acceptable anions, such as chloride, bromide, hydrogen sulfate, methyl sulfate, ethyl sulfate, tetrafluoroborate, phosphate, hydrogen phosphate, dihydrogen phosphate or p-toluene sulfonate, triflate, serve to offset the positive polymer charge of component (c2).

If a methosulfate is used to offset the positive charge of the polymer of formula (Poly3), such N-methyl vinylimidazole/vinylpyrrolidone/vinylimidazole/methacrylamide copolymers are referred to under INCI nomenclature as Polyquaternium-68 and are available, for example, from BASF under the trade name Luviquat® Supreme.

Most particularly preferred copolymers (c3) contain 1 to 12 mol. %, preferably 3 to 9 mol. % and particularly 6 mol % of structural units according to formula (M1-a); 45 to 65 mol %, preferably 50 to 60 mol % and particularly 55 mol % of structural units according to formula (I); 1 to 20 mol %, preferably 5 to 15 mol % and particularly 10 mol % of structural units according to formula (VIII); and 20 to 40 mol %, preferably 25 to 35 mol % and particularly 29 mol % of structural units according to formula (IX).

Particularly preferred agents of this embodiment contain a copolymer (c3) having molar masses within a defined range. Preferred agents are those wherein copolymer (c3) has a molar mass of 100 to 500 kDa, preferably 150 to 400 kDa, more preferably 250 to 350 kDa, and particularly 290 to 310 kDa.

Of the additional film-forming cationic and/or fixing polymer chosen from cationic polymers having at least one structural element of formula (Ml) above, the following are preferred:
N-vinylpyrrolidone/1-vinyl-3-methyl-1H-imidazolium chloride copolymers (such as those having the INCI name Polyquaternium-16 available under the trade names Luviquat® Style, Luviquat® FC 370, Luviquat® FC 550, Luviquat® FC 905 and Luviquat® HM 552 (BASF SE)),
N-vinylpyrrolidone/1-vinyl-3-methyl-1H-imidazolium methyl sulfate copolymers (such as those having the INCI name Polyquaternium-44 available under the trade names Luviquat®Care (BASF SE)),
N-vinylpyrrolidone/N-vinylcaprolactam/1-vinyl-3-methyl-1H-imidazolium terpolymer (such as those having the INCI name Polyquaternium-46 available under the trade names Luviquat® Care or Luviquat® Hold (BASF SE)),
N-vinylpyrrolidone/methacrylamide/N-vinylimidazole/1-vinyl-3-methyl-1H-imidazolium methyl sulfate copolymer (such as those having the INCI name Polyquaternium-68 available under the trade name Luviquat® Supreme (BASF SE)),
   and mixtures of these polymers.

Agents according to the invention can contain as the additional fixing polymer at least one non-ionic fixing polymer. A non-ionic polymer is, according to the invention, a polymer which, in a protic solvent under standard conditions, substantially bears no structural units having permanently cationic or anionic groups needing to be offset by counterions to maintain electrical neutrality. Cationic groups include quaternized ammonium groups but not protonated amines. Anionic groups include carboxyl and sulfonic acid groups.

Non-ionic fixing polymers are preferably present in agents of this embodiment in an amount from 0.1 wt. % to 20.0 wt. % more preferably from 0.2 wt. % to 15.0 wt. %, even more preferably from 0.5 wt. % to 10.0 wt. %, based on total weight of the agent.

Non-ionic fixing polymers having at least one structural element of formula (M2)—

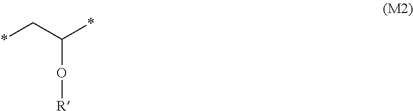

are preferable according to the invention, which have as R′ according to formula (M2) a hydrogen atom, an acetyl group or a propanoyl group, particularly an acetyl group.

Non-ionic fixing polymers are preferably chosen from at least one polymer of—
homopolymers and non-ionic copolymers of N-vinylpyrrolidone, and
non-ionic copolymers of isobutene.

Suitable polyvinyl pyrrolidones include commercial products such as Luviskol® K 90 or Luviskol® K 85 from BASF SE.

Suitable polyvinyl alcohols are sold, for example, under the trade names Elvanol® by Du Pont or Vinol® 523/540 by Air Products.

Suitable polyvinyl acetate is sold, for example, as an emulsion under the trade name Vinac® by Air Products.

Agents that contain as the non-ionic fixing polymer at least one polymer chosen from:
polyvinylpyrrolidone,
copolymers of N-vinylpyrrolidone and vinyl esters of carboxylic acids having 2 to 18 carbon atoms, in particular of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di(C₁ to C₄) alkylamino-(C₂ to C₄)-alkylacrylamide,
copolymers of N-vinylpyrrolidone with N,N-di(C₁ to C₄) alkylamino-(C₂ to C₄)-alkylacrylamide, are most particularly preferred according to the invention.

Preferred agents for treating keratin-containing fibers, particularly human hair, contain in a cosmetically acceptable carrier—
(a) at least one cationic polymer comprising at least one structural unit of formula (I), at least one structural unit of formula (II), and at least one structural unit of formula (III),

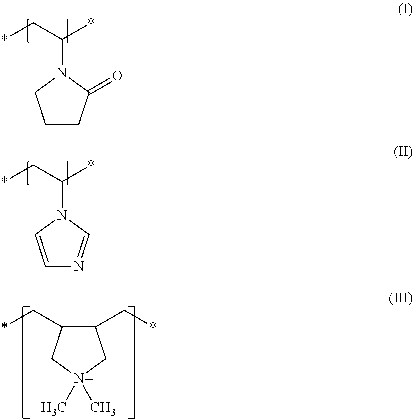

(b) at least one cationic surfactant of formula (IVa)

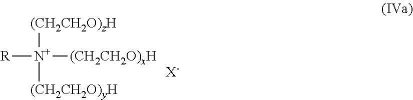

wherein
x, y and z are independently an integer number greater than 0 and the sum of (x+y+z) is a number from 3 to 20, particularly from 5 to 15,
R is a (C₈ to C₂₀) alkyl group or a (C₈ to C₂₀) alkenyl group, and

X⁻ is a physiologically acceptable anion;
and
(c) at least one non-ionic fixing polymer chosen from at least one copolymer of N-vinylpyrrolidone and vinyl esters of carboxylic acids having 2 to 18 carbon atoms (particularly N-vinylpyrrolidone and vinyl acetate).

Furthermore, preferred agents according to the invention for treating keratin-containing fibers, particularly human hair, are those which in a cosmetically acceptable carrier contain—
(a) at least one cationic polymer having at least one structural unit of formula (I), at least one structural unit of formula (II), and at least one structural unit of formula (III),

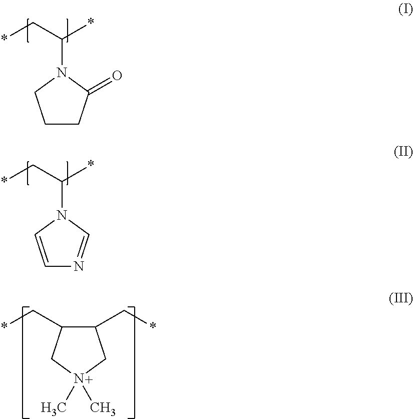

(b) at least one cationic surfactant of formula (IVa)

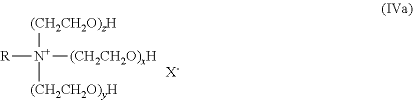

wherein
x, y and z are independently an integer number greater than 0 and the sum of (x+y+z) is a number from 3 to 20, particularly from 5 to 15,
R is a (C₈ to C₂₀) alkyl group or a (C₈ to C₂₀) alkenyl group, and
X⁻ is a physiologically acceptable anion,
and
(c) polyvinylpyrrolidone.

Further possible agents of the embodiment having an additional non-ionic fixing polymer contain as the non-ionic fixing polymer at least one copolymer containing at least one further structural unit according to formula (I) and at least one structural unit according to formula (VII) and at least one structural unit according to formula (VIII)—

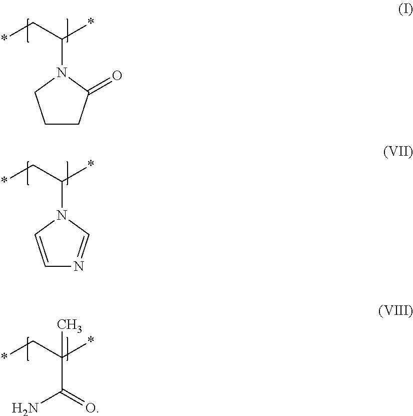

Here, too, these copolymers preferably contain in the copolymer, in addition to polymer units resulting from the incorporation of the cited structural units according to formulae (M1-a), (I), (VII) and (VIII), a maximum of 5 wt. %, preferably a maximum of 1 wt. %, of polymer units resulting from the incorporation of other monomers. Copolymers (c4) are preferably exclusively structural units of formulae (M1-a), (I), (VII) and (VIII) and can be described by the general formula (Poly4)—

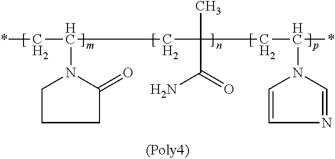

wherein m, n, o and p vary according to the molar mass of the polymer and are not intended to suggest that these are block copolymers. Structural units of formulae (I), (VII) and (VIII) can instead be randomly distributed in the molecule.

A particularly preferred polymer is chosen from polymers with the INCI name VP/Methacrylamide/Vinyl Imidazole Copolymer, obtainable, for example, under the trade name Luviset Clear from BASF SE.

It is particularly preferred for agents according to the invention to additionally contain at least one non-ionic surfactant.

Non-ionic surfactants contain as a hydrophilic group, for example, a polyol group, a polyalkylene glycol ether group or a combination of a polyol and polyglycol ether group. Such compounds include—
addition products of 2 to 100 mol of ethylene oxide and/or 1 to 5 mol of propylene oxide with linear and branched fatty alcohols having 8 to 30 C atoms, with fatty acids having 8 to 30 C atoms and with alkylphenols having 8 to 15 C atoms in the alkyl group;
addition products of 2 to 50 mol of ethylene oxide and/or 1 to 5 mol of propylene oxide with linear and branched fatty alcohols having 8 to 30 C atoms, with fatty acids having 8 to 30 C atoms and with alkylphenols having 8 to 15 C atoms in the alkyl group, end-capped with a methyl or C₂ to C₆ alkyl residue, such as for example the types available under the commercial names Dehydrol® LS, Dehydrol® LT (Cognis);
C₁₂-C₃₀ fatty acid monoesters and diesters of addition products of 1 to 30 mol of ethylene oxide with glycerol;
addition products of 5 to 60 mol of ethylene oxide with castor oil and hydrogenated castor oil;
polyol fatty acid esters, such as the commercial product Hydagen® HSP (Cognis) or Sovermol types (Cognis);
alkoxylated triglycerides;
alkoxylated fatty acid alkyl esters of formula (E4-I)

R¹CO—(OCH₂CHR²)_{w}OR³ (E4-I)

wherein R¹CO is a linear or branched, saturated and/or unsaturated acyl residue having 6 to 22 carbon atoms, R² is hydrogen or methyl, R³ is linear or branched alkyl residues having 1 to 4 carbon atoms, and w is a number from 1 to 20;
amine oxides;
hydroxy mixed ethers, such as are described in DE-OS 19738866;
sorbitan fatty acid esters and addition products of ethylene oxide with sorbitan fatty acid esters such as polysorbates,
sugar fatty acid esters and addition products of ethylene oxide with sugar fatty acid esters;
addition products of ethylene oxide with fatty acid alkanol amides and fatty amines; and
sugar surfactants of the alkyl and alkenyl oligoglycoside type according to formula (E4-II),

R⁴O—[G]ₚ (E4-II)

wherein R⁴ is an alkyl or alkenyl residue having 4 to 22 carbon atoms, G is a sugar residue having 5 or 6 carbon atoms, and p is a number from 1 to 10. They can be obtained by relevant methods of preparative organic chemistry.

Alkylene oxide addition products with saturated linear fatty alcohols and fatty acids each containing 2 to 100 mol of ethylene oxide per mol of fatty alcohol or fatty acid are most particularly preferred non-ionic surfactants. Preparations having outstanding properties are likewise obtained if they contain C₁₂-C₃₀ fatty acid mono- and diesters of addition products of 1 to 30 mol of ethylene oxide with glycerol and/or addition products of 5 to 60 mol of ethylene oxide with castor oil and hydrogenated castor oil as non-ionic surfactants.

Both products having a “normal” homolog distribution and those having a narrow homolog distribution can be used for surfactants that are addition products of ethylene and/or propylene oxide with fatty alcohols or derivatives of these addition products. “Normal” homolog distribution refers to mixtures of homologs obtained by reacting fatty alcohol and alkylene oxide using alkali metals, alkali hydroxides or alkali alcoholates as catalysts. Narrow homolog distributions are obtained when, for example, hydrotalcites, alkaline-earth metal salts of ether carboxylic acids, alkaline-earth metal oxides, hydroxides or alcoholates are used as catalysts. Use of products having a narrow homolog distribution can be preferred.

Agents according to the invention most preferably contain at least one addition product of 15 to 100 mol of ethylene oxide, particularly 15 to 50 mol of ethylene oxide, with a linear or branched (particularly linear) fatty alcohol having 8 to 22 carbon atoms as the surfactant. This is most preferably Ceteareth-15, Ceteareth-25 or Ceteareth-50, which are sold respectively as Eumulgin® CS 15 (COGNIS), Cremophor A25 (BASF SE) and Eumulgin® CS 50 (COGNIS).

Agents according to the invention contain the ingredients and active ingredients in a cosmetically acceptable carrier.

Preferred cosmetically acceptable carriers are aqueous, alcoholic or aqueous-alcoholic media preferably having at least 10 wt. % water, based on total agent. Low alcohols having 1 to 4 carbon atoms that are conventionally used for cosmetic purposes, such as ethanol and isopropanol, can be included in particular as alcohols. It is preferable to use at least one (C₁ to C₄) monoalkyl alcohol in agents according to the invention, particularly in an amount from 1 to 50 wt. %, more particularly from 5 to 30 wt. %. This is particularly preferred for formulation as a pump foam or aerosol foam.

Organic solvents or a mixture of solvents with a boiling point of 400° C. or lower can be included as additional co-solvents in an amount from 0.1 to 15 wt. %, preferably from 1 to 10 wt. %, based on total agent. Unbranched or branched hydrocarbons such as pentane, hexane, and isopentane and cyclic hydrocarbons such as cyclopentane and cyclohexane are particularly suitable as additional co-solvents. Other particularly preferred water-soluble solvents are glycerol, ethylene glycol and propylene glycol in an amount of up to 30 wt. % based on total agent.

In particular, the addition of glycerol and/or propylene glycol and/or polyethylene glycol and/or polypropylene glycol increases the flexibility of the polymer film formed on application of the agent according to the invention. If a flexible hold is desired, the agents according to the invention therefore preferably contain 0.01 to 30 wt. % of glycerol and/or propylene glycol and/or polyethylene glycol and/or polypropylene glycol, relative to the total agent.

The agents preferably have a pH of 2 to 11. The pH range from 2 to 8 is particularly preferred. Unless otherwise specified, within the meaning of this document stated pH values relate to the pH at 25° C.

Agents according to the invention can further contain auxiliary substances and additives that are conventionally added to customary styling agents.

Suitable auxiliary substances and additives include in particular additional care substances.

Silicone oil and/or a silicone gum, for example, can be used as a care substance.

Suitable silicone oils or silicone gums according to the invention include dialkyl and alkylaryl siloxanes, such as dimethyl polysiloxane and methyl phenyl polysiloxane, and the alkoxylated, quaternized or also anionic derivatives thereof. Cyclic and linear polydialkyl siloxanes, the alkoxylated and/or aminated derivatives thereof, dihydroxypolydimethyl siloxanes and polyphenyl alkyl siloxanes are preferred.

Silicone oils give rise to a wide variety of effects. For example, they simultaneously influence the dry and wet compatibility of hair, the feel of dry and wet hair and the gloss. The term silicone oil is understood by one skilled in the art to refer to a plurality of structures of organosilicon compounds. They are firstly understood to be dimethiconols.

The following commercial products are cited as examples of such products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401DC (all of the above Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all of the above Dow Corning Corporation), Dub Gel SI 1400 (Stearinerie Dubois Fils), HVM 4852 Emulsion (Crompton Corporation), Jeesilc 6056 (Jeen International Corporation), Lubrasil, Lubrasil DS (both Guardian Laboratories), Nonychosine E, Nonychosine V (both Exsymol), SanSurf Petrolatum-25, Satin Finish (both Collaborative Laboratories, Inc.), Silatex-D30 (Cosmetic Ingredient Resources), Silsoft 148, Silsoft E-50, Silsoft E-623 (all of the above Crompton Corporation), SM555, SM2725, SM2765, SM2785 (all of the above GE Silicones), Taylor T-Sil CD-1, Taylor TME-4050E (all Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all of the above Wacker-Chemie GmbH).

Dimethicones form the second group of silicones that can be used according to the invention. These can be both linear and branched and also cyclic or cyclic and branched.

Dimethicone copolyols are a further group of silicones that are suitable. Corresponding dimethicone copolyols are commercially available and are sold, for example, by Dow Corning under the name Dow Corning® 5330 Fluid.

The teaching according to the invention naturally also includes the fact that the dimethiconols, dimethicones and/or dimethicone copolymers can already be in the form of an emulsion. The corresponding emulsion of the dimethiconols, dimethicones and/or dimethicone copolyols can be produced both after production of the corresponding dimethiconols, dimethicones and/or dimethicone copolyols and by conventional emulsification methods known to one skilled in the art. To this end, cationic, anionic, non-ionic or zwitterionic surfactants and emulsifiers as auxiliary substances can be used as auxiliary agents to produce the corresponding emulsions. Emulsions of the dimethiconols, dimethicones and/or dimethicone copolyols can also be produced directly by an emulsion polymerization method. Such methods are known to one skilled in the art.

If the dimethiconols, dimethicones and/or dimethicone copolyols are used as an emulsion, then according to the invention the droplet size of the emulsified particles is 0.01 to 10,000 μm, preferably 0.01 to 100 μm, particularly preferably 0.01 to 20 μm and most particularly preferably 0.01 to 10 μm. Particle size is determined by the light scattering method.

If branched dimethiconols, dimethicones and/or dimethicone copolyols are used, this should be understood to mean that the branching is greater than a random branching that occurs by chance due to impurities in the various monomers. Within the meaning of the present invention, branched dimethiconols, dimethicones and/or dimethicone copolyols are therefore understood to have a degree of branching greater than 0.01%. A degree of branching greater than 0.1% is preferred, more preferably greater than 0.5%. The degree of branching is determined from the ratio of unbranched monomers to branched monomers (i.e., the amount of trifunctional and tetrafunctional siloxanes). Both low-branched and highly branched dimethiconols, dimethicones and/or dimethicone copolyols can be most particularly preferred according to the invention.

Particularly suitable silicones are amino-functional silicones, particularly silicones that are grouped together under the INCI name amodimethicones. It is therefore preferable for agents according to the invention additionally to contain at least one amino-functional silicone. These are silicones having at least one, optionally substituted, amino group. Under the INCI declaration, these silicones are known as amodimethicones and are available, for example, in the form of an emulsion as the commercial product Dow Corning® 939 or as the commercial product Dow Corning® 949 mixed with a cationic and a non-ionic surfactant.

Amino-functional silicones are preferably used that have an amine value of 0.25 meq/g or greater, preferably 0.3 meq/g or greater, and more preferably 0.4 meq/g or greater. The amine value is the milli-equivalents of amine per gram of amino-functional silicone. It can be determined by titration and also specified in the unit mg KOH/g.

The agents contain silicones preferably in amounts from 0.01 wt. % to 15 wt. %, more preferably from 0.05 to 2 wt. %, based on total agent.

As a care substance from a different class of compounds, the agent can contain, for example, at least one protein hydrolysate and/or a derivative thereof.

Protein hydrolysates are mixtures of products obtained by acidically, basically or enzymatically catalyzed breakdown of proteins. According to the invention, the term protein hydrolysates also includes total hydrolysates and individual amino acids and derivatives thereof, as well as mixtures of different amino acids. The molecular weight of protein hydrolysates for use according to the invention is from 75, the molecular weight for glycine, to 200,000. The molecular weight is preferably 75 to 50,000 and more preferably 75 to 20,000 Daltons.

According to the invention, protein hydrolysates of plant, animal, marine and/or synthetic origin can be used.

Animal protein hydrolysates include elastin, collagen, keratin, silk and milk protein hydrolysates, which can also be present in the form of salts. Such products are sold, for example, under the trademarks Dehylan® (Cognis), Promois® (Interorgana), Collapuron®(Cognis), Nutrilan® (Cognis), Gelita-Sol® (Deutsche Gelatine Fabriken Stoess & Co), Lexein® (Inolex), Sericin (Pentapharm) and Kerasol® (Croda).

Protein hydrolysates are present in agents according to the invention in amounts, for example, from 0.01 wt. % to 20 wt. %, preferably 0.05 wt. % to 15 wt. % and more preferably from 0.05 wt. % to 5 wt. %, based on total application preparation.

Agents according to the invention can further contain at least one vitamin, provitamin, vitamin precursor and/or derivative thereof as a care substance.

Preferred vitamins, provitamins and vitamin precursors are conventionally assigned to groups A, B, C, E, F and H.

The group of substances classified as vitamin A includes retinol (vitamin A₁) and 3,4-didehydroretinol (vitamin A₂). β-Carotene is the retinol provitamin. Suitable vitamin A components according to the invention are for example vitamin A acid and esters thereof, vitamin A aldehyde and vitamin A alcohol and esters thereof such as the palmitate and acetate. The agents contain the vitamin A component preferably in amounts from 0.05 to 1 wt. %, relative to the total application preparation.

The vitamin B group or the vitamin B complex includes inter alia vitamin B₁ (thiamine), vitamin B₂ (riboflavin), vitamin B₃ (nicotinic acid and nicotinic acid amide (niacinamide)), vitamin B₅ (pantothenic acid, panthenol and pantolactone), vitamin B₆ (pyridoxine as well as pyridoxamine and pyridoxal), vitamin C (ascorbic acid), vitamin E (tocopherols, in particular α-tocopherol), vitamin F (linoleic acid and/or linolenic acid), vitamin H.

Agents according to the invention preferably contain vitamins, provitamins and vitamin precursors from groups A, B, C, E and H. Panthenol, pantolactone, pyridoxine and derivatives thereof as well as nicotinic acid amide and biotin are particularly preferred.

D-Panthenol, optionally in combination with at least one of the aforementioned silicone derivatives, is most particularly preferably used as a care substance.

Like the addition of glycerol and/or propylene glycol, the addition of panthenol increases the flexibility of the polymer film formed on application of the agent according to the invention. If a particularly flexible hold is desired, the agents can therefore contain panthenol instead of or in addition to glycerol and/or propylene glycol. In a preferred embodiment, the agents contain panthenol, preferably in an amount from 0.05 to 10 wt. %, more preferably 0.1 to 5 wt. %, based on total agent.

Agents according to the invention can moreover contain at least one plant extract as a care substance.

These extracts are conventionally produced by extraction of the entire plant. It can also be preferable in individual cases, however, to produce the extracts exclusively from flowers and/or leaves of the plant.

Extracts from green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root are preferred according to the invention.

It can also be preferred to use mixtures of a plurality of different plant extracts, particularly two, in agents according to the invention.

Mono- or oligosaccharides can also be used as a care substance in agents according to the invention.

Both monosaccharides and oligosaccharides, such as cane sugar, lactose and raffinose, can be used. Use of monosaccharides is preferred. Of the monosaccharides, compounds containing 5 or 6 carbon atoms are preferred.

Suitable pentoses and hexoses include ribose, arabinose, xylose, lyxose, allose, altrose, glucose, mannose, gulose, idose, galactose, talose, fucose and fructose. Arabinose, glucose, galactose and fructose are preferably used carbohydrates. Glucose, which is suitable both in the D-(+)- or L-(−)-configuration or as a racemate, is most particularly preferably used.

Derivatives of these pentoses and hexoses, such as the corresponding aldonic and uronic acids (sugar acids), sugar alcohols and glycosides, can moreover also be used according to the invention. Preferred sugar acids are gluconic acid, glucuronic acid, saccharic acid, mannosaccharic acid and mucic acid. Preferred sugar alcohols are sorbitol, mannitol and dulcitol. Preferred glycosides are methyl glucosides.

As mono- or oligosaccharides that are used are conventionally obtained from natural raw materials such as starch, they generally have configurations corresponding to these raw materials (e.g., D-glucose, D-fructose and D-galactose).

The mono- or oligosaccharides are preferably present in the agents in an amount from 0.1 to 8 wt. %, particularly 1 to 5 wt. %, based on total application preparation.

The agent can also contain at least one lipid as a care substance.

Suitable lipids according to the invention are phospholipids, for example, soy lecithin, egg lecithin and cephalins, and substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are sold, for example, by Mona under the commercial names Phospholipid EFA®, Phospholipid PTC® and Phospholipid SV®. Agents according to the invention contain lipids preferably in amounts from 0.01 to 10 wt. %, particularly 0.1 to 5 wt. %, based on total application preparation.

Oil bodies are also suitable as a care substance.

Natural and synthetic cosmetic oil bodies include:
vegetable oils. Examples of such oils are sunflower oil, olive oil, soybean oil, rapeseed oil, almond oil, jojoba oil, orange oil, wheat germ oil, peach kernel oil and the liquid components of coconut butter. Other triglyceride oils are also suitable, such as the liquid components of beef fat and synthetic triglyceride oils.
liquid paraffin oils, isoparaffin oils and synthetic hydrocarbons and also di-n-alkyl ethers having in total 12 to 36 C atoms, particularly 12 to 24 C atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. Commercially available compounds, for example, 1,3-di-(2-ethylhexyl)cyclohexane (Cetiol® S) and di-n-octyl ether (Cetiol® OE), can be preferred.
ester oils. Ester oils are esters of C₆-C₃₀ fatty acids with C₂-C₃₀ fatty alcohols. The monoesters of fatty acids with alcohols having 2 to 24 C atoms are preferred. Particularly preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl)adipate, di-(2-ethylhexyl)succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate;
symmetrical, asymmetrical or cyclic esters of carbonic acid with fatty alcohols, as described, for example, in DE-OS 197 56 454, glycerol carbonate or dicaprylyl carbonate (Cetiol® CC);
tri-fatty acid esters of saturated and/or unsaturated linear and/or branched fatty acids with glycerol;
fatty acid partial glycerides, namely monoglycerides, diglycerides and technical mixtures thereof. If technical products are used, small amounts of triglycerides may also be included for production reasons. The partial glycerides are preferably of the formula (D4-I),

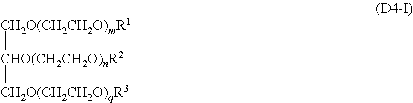

wherein R¹, R² and R³ are independently hydrogen or a linear or branched, saturated and/or unsaturated acyl residue having 6 to 22, preferably 12 to 18, carbon atoms, with the proviso that at least one of these groups is an acyl residue and at least one of these groups is hydrogen. The sum (m+n+q) is 0 or a number from 1 to 100, preferably 0 or 5 to 25. R¹ is preferably an acyl residue, R² and R³ hydrogen, and the sum (m+n+q) is preferably 0. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Oleic acid monoglycerides are preferably used.

The amount of natural and synthetic cosmetic oil bodies used in agents according to the invention is conventionally 0.1 to 30 wt. %, preferably 0.1 to 20 wt. %, and particularly 0.1 to 15 wt. %, based on total application preparation.

Although each of the specified care substances individually gives rise to a satisfactory result, all embodiments in which the agent contains a plurality of care substances, including examples from different groups, are also included in the context of the present invention.

With the addition of a UV filter, both the agents and the treated fibers can be protected from damaging influences of UV radiation. At least one UV filter is therefore preferably added to the agent. There are no general restrictions on suitable UV filters in terms of their structure and physical properties. In fact, all UV filters that can be used in the cosmetics sector whose absorption maximum is in the UVA (315-400 nm), UVB (280-315 nm) or UVC (<280 nm) range are suitable. UV filters having an absorption maximum in the UVB range, particularly in the range from approximately 280 to approximately 300 nm, are particularly preferred.

Preferred UV filters according to the invention include substituted benzophenones, p-aminobenzoic acid esters, diphenyl acrylic acid esters, cinnamic acid esters, salicylic acid esters, benzimidazoles and o-aminobenzoic acid esters.

Examples of UV filters that can be used according to the invention are 4-aminobenzoic acid, N,N,N-trimethyl-4-(2-oxoborn-3-ylidene methyl)aniline methyl sulfate, 3,3,5-trimethyl cyclohexyl salicylate (Homosalate), 2-hydroxy-4-methoxybenzophenone (Benzophenone-3; Uvinul® M 40, Uvasorb® MET, Neo Heliopan® BB, Eusolex® 4360), 2-phenylbenzimidazole-5-sulfonic acid and potassium, sodium and triethanolamine salts thereof (Phenylbenzimidazole sulfonic acid; Parsol® HS; Neo Heliopan® Hydro), 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione (Butyl methoxydibenzoylmethane; Parsol® 1789, Eusolex® 9020), α-(2-oxoborn-3-ylidene)toluene-4-sulfonic acid and salts thereof, ethoxylated 4-aminobenzoic acid ethyl ester (PEG-25 PABA; Uvinul® P 25), 4-dimethylaminobenzoic acid-2-ethylhexyl ester (Octyl Dimethyl PABA; Uvasorb® DMO, Escalol® 507, Eusolex® 6007), salicylic acid-2-ethylhexyl ester (Octyl Salicylate; Escalol® 587, Neo Heliopan® OS, Uvinul® O18), 4-methoxycinnamic acid isopentyl ester (Isoamyl p-Methoxycinnamate; Neo Heliopan® E 1000), 4-methoxycinnamic acid-2-ethylhexyl ester (Octyl Methoxycinnamate; Parsol® MCX, Escalol557, Neo Heliopan® AV), 2-hydroxy-4-methoxybenzophenone-5-sulfonic acid and the sodium salt thereof (Benzophenone-4; Uvinul® MS 40; Uvasorb® S 5), 3-(4′-methylbenzylidene)-D,L-camphor (4-Methylbenzylidene camphor; Parsol® 5000, Eusolex® 6300), 3-benzylidene camphor (3-Benzylidene camphor), 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)-[2-oxoborn-3-ylidene methyl]benzyl}acrylamide, 2,4-dihydroxybenzophenone (Benzophenone-1; Uvasorb® 20 H, Uvinul® 400), 1,1′-diphenylacrylonitrilic acid-2-ethylhexyl ester (Octocrylene; Eusolex® OCR, Neo Heliopan® Type 303, Uvinul® N 539 SG), o-aminobenzoic acid menthyl ester (Menthyl Anthranilate; Neo Heliopan® MA), 2,2′,4,4′-tetrahydroxybenzophenone (Benzophenone-2; Uvinul® D-50), 2,2′-dihydroxy-4,4′-dimethoxybenzophenone (Benzophenone-6), 2,2′-dihydroxy-4,4′-dimethoxybenzophenone-5-sodium sulfonate and 2-cyano-3,3-diphenylacrylic acid-2′-ethylhexyl ester. 4-Aminobenzoic acid, N,N,N-trimethyl-4-(2-oxoborn-3-ylidene methyl)aniline methyl sulfate, 3,3,5-trimethyl cyclohexyl salicylate, 2-hydroxy-4-methoxybenzophenone, 2-phenylbenzimidazole-5-sulfonic acid and potassium, sodium and triethanolamine salts thereof, 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione, α-(2-oxoborn-3-ylidene)toluene-4-sulfonic acid and salts thereof, ethoxylated 4-aminobenzoic acid ethyl ester, 4-dimethylaminobenzoic acid-2-ethylhexyl ester, salicylic acid-2-ethylhexyl ester, 4-methoxycinnamic acid isopentyl ester, 4-methoxycinnamic acid-2-ethylhexyl ester, 2-hydroxy-4-methoxybenzophenone-5-sulfonic acid and the sodium salt thereof, 3-(4′-methylbenzylidene)-D,L-camphor, 3-benzylidene camphor, 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)-[2-oxoborn-3-ylidene methyl]benzyl}acrylamide are preferred. Most particularly preferred according to the invention are 2-hydroxy-4-methoxybenzophenone, 2-phenylbenzimidazole-5-sulfonic acid and potassium, sodium and triethanolamine salts thereof, 1-(4-tert-butylphenyl)-3-(4-methoxyphenyl)propane-1,3-dione, 4-methoxycinnamic acid-2-ethylhexyl ester and 3-(4′-methylbenzylidene)-D,L-camphor.

UV filters are conventionally included in amounts from 0.01 to 5 wt. %, based on total application preparation. Amounts from 0.1 to 2.5 wt. % are preferred.

In a special embodiment, agents according to the invention also contain one or more substantive dyes. This allows the treated keratinic fibers not only to be temporarily structured but at the same time also be colored through use of the agent. This can be particularly desirable if only a temporary coloration is desired, for example, with striking fashion colors, which can be removed again from the keratinic fibers simply by washing.

Substantive dyes are conventionally nitrophenylene diamines, nitroaminophenols, azo dyes, anthraquinones or indophenols. Preferred substantive dyes are the compounds known under the international names or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 11, HC Blue 12, Disperse Blue 3, Acid Blue 7, Acid Green 50, HC Violet 1, Disperse Violet 1, Disperse Violet 4, Acid Violet 43, Disperse Black 9, Acid Black 1 and Acid Black 52 as well as 1,4-diamino-2-nitrobenzene, 2-amino-4-nitrophenol, 1,4-bis-(1-hydroxyethyl)-amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitro-diphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-1-hydroxy-4-nitrobenzene. Cationic substantive dyes are preferably used. Of those, the following are particularly preferred:
(a) cationic triphenylmethane dyes, such as Basic Blue 7, Basic Blue 26, Basic Violet 2 and Basic Violet 14;
(b) aromatic systems substituted with a quaternary nitrogen group, such as Basic Yellow 57, Basic Red 76, Basic Blue 99, Basic Brown 16 and Basic Brown 17; and
(c) substantive dyes containing a heterocyclic compound having at least one quaternary nitrogen atom, such as those mentioned in EP-A2-998 908, for example, in claims 6 to 11 therein.

Dyes known under the names Basic Yellow 87, Basic Orange 31 and Basic Red 51 are particularly preferred cationic substantive dyes of group (c). Cationic substantive dyes sold under the trademark Arianor® are likewise particularly preferred cationic substantive dyes according to the invention.

Agents according to this embodiment preferably contain substantive dyes in an amount from 0.001 to 20 wt. %, based on total agent.

Preferably, agents according to the invention are free from oxidation dye precursors. Oxidation dye precursors are divided into developer components and coupler components. The developer components form the actual dyes under the influence of oxidizing agents or of atmospheric oxygen with one another or by coupling with one or more coupler components.

Agents according to the invention can be formulated in all conventional forms for styling agents, for example, as solutions, which can be applied to the hair as a hair lotion or pump or aerosol spray, as creams, emulsions, waxes, gels or surfactant-containing foaming solutions or other preparations suitable for application on the hair.

Hair creams and hair gels generally contain structuring agents and/or thickening polymers, which serve to impart the desired consistency to the products. Structuring agents and/or thickening polymers are typically used in an amount from 0.1 to 10 wt. %, based on total product. Amounts from 0.5 to 5 wt. %, particularly 0.5 to 3 wt. %, are preferred.

Agents according to the invention are preferably formulated as a pump spray, aerosol spray, pump foam, or aerosol foam.

To this end, the agents are presented in a dispensing device, which is either a compressed gas cylinder (aerosol container) additionally filled with a propellant or a non-aerosol container.

Compressed gas cylinders, by which a product is distributed through a valve via the internal gas pressure in the cylinder, are referred to as “aerosol containers”. In contrast to the aerosol container, a “non-aerosol container” is a container under normal pressure, by which a product is distributed through a pump system by a mechanical action.

Agents according to the invention are preferably formulated as an aerosol hair foam or aerosol hair spray. The agent therefore preferably additionally contains at least one propellant.

Suitable propellants according to the invention include N₂O, dimethyl ether, CO₂, air, alkanes having 3 to 5 carbon atoms, such as propane, n-butane, isobutane, n-pentane and isopentane, and mixtures thereof. Dimethyl ether, propane, n-butane, isobutane and mixtures thereof are preferred.

In a preferred embodiment, the specified alkanes, mixtures of the specified alkanes or mixtures of the specified alkanes with dimethyl ether are used as the sole propellant. However, the invention also includes the incorporation of chlorofluorocarbon propellants, particularly fluorocarbons.

In the given spray device, the size of the aerosol droplets or foam bubbles and the size distribution can each be adjusted by the ratio of propellant to the other constituents of the preparations.

The amount of propellant used varies according to the specific composition of the agent, the packaging used and the desired product type (e.g., hair spray or hair foam). If conventional spray devices are used, aerosol foam products preferably contain propellant in an amount from 1 to 35 wt. %, based on total product. Amounts from 2 to 30 wt. %, particularly 3 to 15 wt. %, are particularly preferred. Aerosol sprays generally contain larger amounts of propellant. Here, the propellant is preferably used in an amount from 30 to 98 wt. %, based on total product. Amounts from 40 to 95 wt. %, particularly 50 to 95 wt. %, are particularly preferred.

The aerosol products can be produced using conventional techniques. All ingredients of the individual agent except for the propellant are generally introduced into a suitable pressure-resistant container. This is then closed with a valve. Finally, the desired amount of propellant is added using conventional techniques.

Isopentane is preferably suitable as a propellant for expanding agents in gel form in a two-chamber aerosol container, it being incorporated into agents according to the invention and introduced into the first chamber of the two-chamber aerosol container. At least one further propellant different from isopentane and that establishes a higher pressure in the two-chamber aerosol container than isopentane is introduced into the second chamber of the two-chamber aerosol container. The propellants of the second chamber are preferably chosen from N₂O, dimethyl ether, CO₂, air, alkanes having 3 or 4 carbon atoms (such as propane, n-butane, isobutane) and mixtures thereof.

Aerosol hair foams or aerosol hair sprays containing agents as described above and at least one propellant are a preferred embodiment of agents according to the invention.

Preferred agents according to the invention and propellants of the aerosol hair foam or aerosol hair spray and the amounts of propellant correspond to the details specified above.

The invention secondly provides for use of agents according to the invention for temporarily shaping hair and/or for hair care.

Agents according to the invention and products containing these agents, particularly aerosol hair foams or aerosol hair sprays, impart a very strong, lasting styling hold to the treated hair, even though the hair remains flexible. If the agent is formulated as hair foam, a stable, fine-pored and creamy foam is formed which can be distributed evenly onto the hair without dripping.

The invention thirdly provides a method for treating keratin-containing fibers, particularly human hair, wherein an agent according to the first subject-matter of the invention is expanded into foam using a dispensing device and the resulting foam applied to the keratin-containing fibers.

Preferably, the keratin-containing fibers are shaped and this shape is fixed by the agent of the first subject-matter of the invention.

The aforementioned dispensing devices (see above) are preferred according to the invention.

The invention fourthly provides a method for treating keratin-containing fibers, particularly human hair, wherein, using a dispensing device, an agent according to the first subject-matter of the invention is applied to the keratin-containing fibers as a spray.

It is preferable for the keratin-containing fibers to be shaped and for this shape to be fixed by the agent of the first subject-matter of the invention.

The aforementioned dispensing devices (see above) are preferred according to the invention.

The examples below are intended to illustrate the subject-matter of the present invention without in any way limiting it.

### EXAMPLES

Unless otherwise specified, the quantities given below are percentages by weight.

The following formulations were prepared:

| | E1 | E2 | E3 | E4 | E5 |
|---|---|---|---|---|---|
| PVP/VA 64 W¹ | — | 10.0 | — | — | — |
| Gafquat ® 755N | — | — | 8.0 | — | — |
| Luviquat ® Sensation | 5.0 | 0.3 | 0.3 | 0.3 | 0.3 |
| Styleze W10² | — | — | — | 5.0 | 3.0 |
| Aquastyle 300³ | — | — | — | 5.0 | 3.0 |
| Celquat L200⁴ | — | — | — | — | 1.0 |
| Dehyquart ® SP | 0.5 | 0.5 | 0.5 | 0.5 | 0.5 |
| Perfume | 0.2 | 0.2 | 0.2 | 0.2 | 0.2 |
| Propane/butane⁵ | 6.0 | 6.0 | 6.0 | 6.0 | 6.0 |
| Water | 83.0 | 83.0 | 85.0 | 83.0 | 86.0 |

¹Copolymer of vinyl acetate and N-vinylpyrrolidone in the ratio 40 to 60 (50% active substance in water, INCI name: VP/VA Copolymer) (BASF);
²Copolymer of methacryloylaminopropyl lauryl dimethylammonium chloride with N-vinylpyrrolidone and dimethylaminopropyl methacrylamide with the INCI name Polyquaternium-55 (10 wt. % active substance in water) (ISP);
³Copolymer of methacryloylaminopropyl lauryl dimethylammonium chloride with N-vinylpyrrolidone, N-vinylcaprolactam and dimethylaminopropyl methacrylamide with the INCI name Polyquaternium-69 (30 wt. % active substance in water) (ISP);
⁴Quaternized cellulose derivative (INCI name: Polyquaternium-4) (National Starch);
⁵Propane/butane mixture (47 wt. % propane, 50 wt. % butane, 3 wt. % isobutane).

The formulation ingredients for each formulation were introduced into separate aerosol containers that satisfies the following technical parameters: aluminum storage vessel with valve, product 522983 PV10697 from Precision (Deutsche Prazisions-Ventil GmbH).

Each formulation was dispensed from the aerosol containers as hair foam and distributed on the hair of a test subject. The formulations were perceived to be gentle on the skin and, when applied to the hair, brought about a styling hold. A very good care effect on the hair was obtained.

We claim:

1. An agent for treating keratin-containing fibers comprising in a cosmetically acceptable carrier:
(a) at least one cationic polymer encompassing at least one structural unit of formula (I), at least one structural unit of formula (II), and at least one structural unit of formula (III), 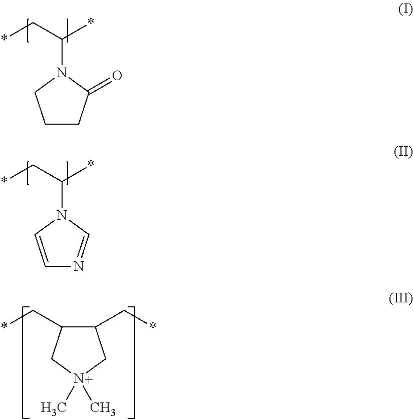
(b) at least one cationic surfactant of formula (IV) 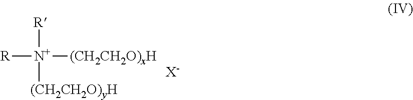
wherein
x and y are independently an integer greater than 0,
R is a (C₈ to C₂₀) alkyl group or a (C₈ to C₂₀) alkenyl group,
R′ is a *—(CH₂CH₂O)_{z}H group wherein z is an integer greater than 0, a (C₈ to C₂₀) alkyl group, or a (C₈ to C₂₀) alkenyl group, and
X⁻ is a physiologically acceptable anion.

2. The agent according to claim 1, wherein the at least one cationic polymer has an average molecular weight M_{w} (weight average) from 50,000 to 250,000 g/mol.

3. The agent according to claim 1, wherein at least one cationic polymer is at least a polymer having the INCI name Polyquaternium-87.

4. The agent according to claim 1, wherein the at least one cationic polymer is present in an amount from 0.01 wt. % to 20 wt. %, based on total weight of the agent.

5. The agent according to claim 1, wherein the cationic surfactant of formula (IV) is present in an amount from 0.01 wt. % to 20 wt. %, based on total weight of the agent.

6. The agent according to claim 1, wherein the cationic surfactant of formula (IV) has a molecular weight from 550 to 2500 g/mol.

7. The agent according to claim 1, wherein the sum of x+y according to formula (IV) is a number from 3 to 20.

8. The agent according to claim 1, wherein R′ according to formula (IV) is a *—(CH₂CH₂O)_{z}H group wherein z is an integer greater than 0.

9. The agent according to claim 8, wherein the sum of x+y+z is a number from 3 to 20.

10. An aerosol foam or aerosol spray comprising the agent according claim 1.

11. The agent according to claim 1 further comprising at least one fixing polymer.

12. A method for treating keratin-containing fibers comprising expanding an agent according to claim 1 into a foam using a dispensing device, and applying the resulting foam to the keratin-containing fibers.

13. A method for treating keratin-containing fibers comprising applying an agent according to claim 1 as a spray to the keratin-containing fibers using a dispensing device.
